(19) United States
(12) Patent Application Publication
(10) Pub. No.: US 2026/0007699 A1
(43) Pub. Date: Jan. 8, 2026

(54) COMPOSITIONS AND METHODS FOR TREATING GLUTEN-RELATED DISORDERS

(71) Applicants: Beth Israel Deaconess Medical Center, Inc., Boston, MA (US); Milky Way Life Sciences LLC, Cambridge, MA (US)

(72) Inventors: Amelie Therrien, Brookline, MA (US); Ciaran Patrick Kelly, West Newton, MA (US); Jocelyn Anne Silvester, Boston, MA (US); Xinhua Chen, Cambridge, MA (US); Weishu Zhu, Quincy, MA (US)

(21) Appl. No.: 18/993,340

(22) PCT Filed: Jul. 12, 2023
(86) PCT No.: PCT/US2023/027522

§ 371 Date: Jan. 10, 2025

Related U.S. Application Data
(60) Provisional application No. 63/388,503, filed on Jul. 12, 2022.

(51) Int. Cl. A61K 35/20; A61K 9/00; A61K 39/00; A61K 39/35; A61P 1/14; C07K 16/16

(52) U.S. Cl. CPC A61K 35/20; A61K 9/0053; A61K 39/35; A61P 1/14; C07K 16/16; A61K 2039/54; A61K 2039/545

(57) ABSTRACT

Provided are compositions and methods for alleviating symptoms or treating gluten-related diseases. In some embodiments, the compositions include colostrum from cows that are immunized with gluten or partially digested gluten.

### CLAIM OF PRIORITY

This application claims the benefit of U.S. Provisional Application 63/388,503, filed on Jul. 12, 2022. The entire contents of the foregoing are incorporated herein by reference.

### TECHNICAL FIELD

This disclosure relates to compositions and methods for alleviating symptoms or treating gluten-related diseases.

### BACKGROUND

Gluten is a general name for the proteins found in wheat (wheatberries, durum, emmer, semolina, spelt, farina, farro, graham, KAMUT® khorasan wheat and einkorn), rye, barley and triticale. Gluten helps foods maintain their shape, acting as a glue that holds food together. Gluten can be found in many types of foods, even ones that would not be expected.

Many people have gluten-related disorders, e.g., celiac disease (CD), non-celiac gluten sensitivity (NCGS), gluten ataxia, dermatitis herpetiformis (DH) and wheat allergy. There is an urgent need to treat, reduce the risk of developing, and/or alleviating symptoms of gluten-related disorders.

### SUMMARY

This disclosure relates to hyperimmune bovine colostrum to block absorption of gliadin peptides in the human intestine. Gliadin is a prolamin (protein) present in wheat that does not get fully broken down to amino acids by human digestive enzymes. Therefore, even after digestion by pepsin and trypsin (PT-gliadin), gliadin polypeptides in the gastrointestinal lumen are toxic for people with celiac disease or non-celiac gluten/wheat sensitivity. Hence, provided herein is a vaccine containing this peptide, to stimulate the formation of immunoglobulins specific to these peptides in cows, and a composition comprising an immunoglobulin that specifically binds to gluten or partially digested gluten.

In one aspect, the disclosure is related to a composition comprising colostrum, in some embodiments, the colostrum comprises an immunoglobulin that specifically binds to gluten or partially digested gluten. In some embodiments, the colostrum is from a non-human mammal. In some embodiments, the colostrum is from a cow (e.g., black Holstein, brown Holstein, or Jersey), buffalo, goat, reindeer, horse, sheep, camel, or yak. In some embodiments, the gluten or partially digested gluten is gliadin or pepsin-trypsin resistant gliadin (PT-gliadin). In some embodiments, the composition is for alleviating symptoms of gluten-related disorder or treating gluten-related disorder in a subject. In some embodiments, the subject is a human. In some embodiments, the gluten-related disorder is celiac disease, non-celiac gluten sensitivity (NCGS), or gluten intolerance. In some embodiments, the immunoglobulin is IgG. In some embodiments, the immunoglobulin has a concentration of at least 1 mg/mL, at least 2 mg/mL, at least 3 mg/mL, at least 4 mg/mL, at least 5 mg/mL, at least 6 mg/mL, at least 7 mg/mL, at least 8 mg/mL, at least 9 mg/mL, at least 10 mg/mL, at least 15 mg/mL, at least 20 mg/mL, at least 25 mg/mL, at least 30 mg/mL, at least 35 mg/mL, at least 40 mg/mL, at least 45 mg/mL, or at least 50 mg/mL in the colostrum. In some embodiments, the composition can inhibit or interfere absorption of the gluten or partially digested gluten in gastrointestinal (GI) tract of the subject. In some embodiments, the immunoglobulin that specifically binds to the gluten or partially digested gluten is about 1-fold, about 2-fold, about 3-fold, about 4-fold, about 5-fold, about 6-fold, about 7-fold, about 8-fold, about 9-fold, about 10-fold, about 15-fold, about 20-fold, about 30-fold, about 40-fold, about 50-fold, about 60-fold, about 70-fold, about 80-fold, about 90-fold, or about 100-fold higher than that in a reference non-colostrum milk sample, or that in a reference colostrum sample that is produced by an animal without being immunized by the gluten or partially digested gluten. In some embodiments, the composition is a lyophilized composition.

In one aspect, the disclosure is related to a food (e.g., medicinal food) or food additive prepared from the composition as described herein.

In one aspect, the disclosure is related to a method of immunizing an animal for production of colostrum, in some embodiments, the colostrum comprises an immunoglobulin that specifically binds to gluten or partially digested gluten, the method comprises: a) administering the gluten or partially digested gluten to the animal, optionally with an adjuvant; and b) collecting one or more milkings from the animal after parturition, in some embodiments, at least one of the one or more milkings comprises the colostrum. In some embodiments, the animal is a non-human mammal. In some embodiments, the animal is pregnant when administered with the gluten or partially digested gluten. In some embodiments, the animal is a cow (e.g., black Holstein, brown Holstein, or Jersey), buffalo, goat, reindeer, horse, sheep, camel, or yak. In some embodiments, the animal is a cow. In some embodiments, the animal is injected at least once, at least twice, at least three times, at least four times, at least five times, or at least six times prior to parturition. In some embodiments, the administering the gluten or partially digested gluten to the animal occurs at about 15 weeks, about 14 weeks, about 13 weeks, about 12 weeks, about 11 weeks, about 10 weeks, about 9 weeks, about 8 weeks, about 7 weeks, about 6 weeks, or about 5 weeks prior to parturition. In some embodiments, the collecting one or more milkings occurs within about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days about 4 days, about 5 days, about 6 days, or about 7 days after parturition.

In one aspect, the disclosure is related to a method of alleviating symptoms of a gluten-related disorder in a subject, comprising administering an effective amount of the composition as described herein, or the food or food additive as described herein, to the subject.

In one aspect, the disclosure is related to a method for treating a gluten-related disorder in a subject, comprising administering an effective amount of the composition as described herein, or the food or food additive as described herein, to the subject. In some embodiments, the composition, the food, or the food additive is orally administered. In some embodiments, the composition, the food, or the food additive is orally administered with other food to the subject. In some embodiments, the composition, the food, or the food additive is orally administered to the subject before eating other food. In some embodiments, the other food comprises gluten. In some embodiments, the subject is a human. In some embodiments, the gluten-related disorder is celiac disease, non-celiac gluten sensitivity (NCGS), or gluten intolerance. In some embodiments, the method described herein further comprises providing gluten-free diet to the subject.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schema of the immunization schedule and blood collection.

FIGS. 2A-2B show optical density for PT-gliadin IgG per ELISA according to the time elapsed since first immunization. The vertical dotted lines represent immunization days. The horizontal dotted line represents the baseline OD pre-immunization. Day 0 represents the day of first immunization.

FIG. 3 shows optical density for PT-gliadin IgG per ELISA according to the milking.

FIG. 4 shows the correlation between the OD readings of the colostrum ELISA results (first milking) and serum (post second booster).

FIG. 5 shows trends of circulating anti-PT-gliadin antibodies in the serum pre, per and post immunization according to the OD readings of the colostrum ELISA results. The arrow indicates a representative cow.

FIG. 6 shows OD readings of PT-gliadin IgG according to ELISA in liquid colostrum and after undergoing lyophilisation (freeze-dry).

FIG. 7 shows OD readings of ELISA results of PT-gliadin IgG in unprocessed and processed colostrum.

FIG. 8A shows the standard curve of OD readings obtained from ELISA testing of unbound PT-gliadin, plotted against the concentration of the loaded PT-gliadin.

FIGS. 8B-8D show the binding capacity of hyperimmune colostrum #1, hyperimmune colostrum #2, and the mix of 3 hyperimmune colostrum to PT-gliadin at different combining ratios.

### DETAILED DESCRIPTION

Celiac disease is a T cell-mediated enteropathy for which the inciting antigen is gluten. Gluten is a group of proteins present in wheat, rye and barley, among which gliadin from wheat is the most frequent gluten protein in the Western diet. Currently, the only available treatment for celiac disease is a strict gluten-free diet. A gluten-free diet may enable the intestine to recover to normal. However, in practice this is difficult to achieve. Completely eliminating all gluten from the diet is extremely challenging because gluten is present in many foods. Further, ostensibly gluten-free foods are easily contaminated during preparation or serving. Moreover, refusing food or eating different food to the rest of the group may be considered impolite and can be socially stigmatizing. Thus, many patients with celiac disease have persistent mucosal damage despite trying to follow a strict gluten-free diet. The majority report accidental gluten exposures which most commonly occur in situations where the person with celiac disease must rely upon others to prepare their food, such as at a restaurant, workplace, cafeteria, at other peoples' homes or when travelling.

In the United States and in Europe, millions of individuals who do not have celiac disease also follow a gluten-free diet. It has been estimated that about 300,000 patients in the United States follow a gluten-free diet (over 75% of those with celiac disease are undiagnosed and so remain on a gluten-containing diet). While some are on a gluten-free diet for celiac disease, many more are following a low gluten or gluten-free diet for other reasons, including to mitigate symptoms of “non-celiac gluten sensitivity” (NCGS). These symptoms overlap the symptoms of celiac disease and may be precipitated by exposure to very small amounts of gluten. Thus, the challenges and concerns described above for celiac patients attempting to follow a gluten-free diet also pertain to those with NCGS. Even small amounts of gluten (e.g., equivalent to the size of breadcrumbs) may be toxic for a patient with celiac disease or NCGS.

Up to 10% of the population avoids gluten but does not have a celiac disease. The diagnosis of having symptoms upon gluten exposure, in the absence of celiac disease and wheat allergy, that resolve with a gluten-free diet is named non-celiac gluten sensitivity (NCGS). This condition entails more extra-intestinal manifestations than the celiac disease. Even though this condition has a high impact on individual's quality of life, because duodenal biopsies are deemed normal and there is no malabsorption or long-term organic consequences, no treatments are on the horizon for NCGS other than the gluten-free diet.

There is a clear need for adjunct treatments to a gluten-free diet to prevent the harmful effects of accidental gluten ingestion. One strategy is to prevent the absorption of gluten so that it cannot stimulate the immune system. Indeed, gluten is partially digested by digestive enzymes and long peptides are able to cross the epithelial barrier into the lamina propria. In celiac disease, tissue transglutaminase deamidates the glutamine amino acids in gluten which changes the peptides configuration, so it fits the HLADQ2 or DQ8 binding groove on antigen presenting cells. Finally, antigen-specific T cell receptors trigger a T cell response. Above all, given the relatively high safety of a gluten-free diet, any adjunct therapy must have an equally highly favorable safety profile.

Recently, anti-gliadin IgY antibodies from eggs produced by hens after immunizing with gliadin were shown to reduce the absorption and toxicity of gliadin in the human intestines. Interestingly, bovine milk naturally contains immunoglobulin G and other components that may have a role in immune regulation (e.g., short chain fatty acids, TGF-β, IL-10). Moreover, hyperimmune colostrum is a technique that has been studied for decades for immune exclusion and immune tolerance for allergens. Specific antibodies targeting infectious agents have been shown to help prevent or treat multiple diseases, e.g., HIV induced enteropathy, rotavirus, E. coli, and C. difficile infections. These IgG molecules are produced by immunizing cows during their pregnancies and are present at maximal concentrations in the colostrum (e.g., first milkings after calving).

Thus, hyperimmune bovine colostrum containing gliadin-specific immunoglobulins would have many properties of an ideal gliadin-binding agent. Bovine IgG can be produced in large quantities at relatively low cost to satisfy demand. Polyclonal antibodies also provide advantages because they can recognize the multiple gliadin epitopes which are immunogenic for patients with celiac disease.

### Gluten and Gluten-Related Disorders

Gluten is a structural protein naturally found in certain cereal grains. It includes the combination of prolamin and glutelin proteins naturally occurring in all grains that have been proved to be capable of triggering celiac disease. These include any species of wheat (e.g., common wheat, durum, spelt, khorasan, emmer and einkorn), barley, rye and some oat cultivars, as well as any cross hybrids of these grains (such as triticale). Gluten makes up 75-85% of the total protein in bread wheat. In some embodiments, the gluten protein is from wheat. In some embodiments, the gluten protein is from barley (secalin) or rye (hordein).

Glutens, especially Triticeae glutens, have unique viscoelastic and adhesive properties, which give dough its elasticity, helping it rise and keep its shape and often leaving the final product with a chewy texture. These properties, and its relatively low cost, make gluten valuable to both food and non-food industries.

Wheat gluten is composed of mainly two types of proteins: the glutenins and the gliadins, which in turn can be divided into high molecular and low molecular glutenins and α/β, γ and Ω gliadins. Its homologous seed storage proteins in barley, are referred to as hordeins; in rye, secalins; and in oats, avenins. These protein classes are collectively referred to as gluten. The storage proteins in other grains, such as maize (zeins) and rice (rice protein), are sometimes called gluten, but they do not cause harmful effects in people with celiac disease.

Gluten can trigger adverse inflammatory, immunological and autoimmune reactions in some people. The spectrum of gluten related disorders includes celiac disease in 1-2% of the general population, non-celiac gluten sensitivity in 0.5-13% of the general population, as well as dermatitis herpetiformis, gluten ataxia and other neurological disorders. These disorders can be treated by a gluten-free diet. Thus, a “gluten-related disorder” refers to a disorder in a subject (e.g., human) that is directly (e.g., via its toxic effect to GI tract) or indirectly (e.g., neurological or psychiatric disorders) caused by gluten or partially digested gluten.

In some embodiments, the gluten described herein is from wheat, barley, Rye, triticale, or oats. In some embodiments, the gluten related disorder is celiac disease, non-celiac gluten sensitivity (NCGS), gluten ataxia, dermatitis herpetiformis (DH) or wheat allergy.

### Celiac Disease

Celiac disease (CD) is a chronic, multiple-organ autoimmune disorder primarily affecting the small intestine caused by the ingestion of wheat, barley, rye, oats, and derivatives, that appears in genetically predisposed people of all ages. CD is not only a gastrointestinal disease, because it may involve several organs and cause an extensive variety of non-gastrointestinal symptoms, and most importantly, it may be apparently asymptomatic. Many asymptomatic people become accustomed to living with a chronic bad health status as if it were normal, but they are able to recognize that they actually had symptoms related to celiac disease after starting a gluten-free diet and improvement occurs. Added difficulties for diagnosis are the fact that serological markers (anti-tissue transglutaminase) are not always present and many people may have minor mucosal lesions, without atrophy of the intestinal villi.

CD affects approximately 1-2% of the general population, but most cases remain unrecognized, undiagnosed and untreated, and at risk for serious long-term health complications. People may suffer severe disease symptoms and be subjected to extensive investigations for many years, before a proper diagnosis is achieved. Untreated CD may cause malabsorption, reduced quality of life, iron deficiency, osteoporosis, an increased risk of intestinal lymphomas, and greater mortality. CD is associated with some other autoimmune diseases, such as diabetes mellitus type 1, thyroiditis, gluten ataxia, psoriasis, vitiligo, autoimmune hepatitis, dermatitis herpetiformis, primary sclerosing cholangitis, and more.

CD with “classic symptoms”, which include gastrointestinal manifestations such as chronic diarrhea and abdominal distention, malabsorption, loss of appetite, and impaired growth, is currently the least common presentation form of the disease and affects predominantly small children generally younger than two years of age. CD with “non-classic symptoms” is the most common clinical type and occurs in older children (over 2 years old), adolescents, and adults. It is characterized by milder or even absent gastrointestinal symptoms and a wide spectrum of non-intestinal manifestations that can involve any organ of the body, and very frequently may be completely asymptomatic both in children (at least in 43% of the cases) and adults.

Asymptomatic CD (ACD) may be present in the majority of affected patients and is characterized by the absence of classical gluten-intolerance signs, such as diarrhea, bloating, and abdominal pain. Nevertheless, these individuals very often develop diseases that can be related with gluten intake. Gluten can be degraded into several morphine-like substances, named gluten exorphins.

### Non-Celiac Gluten Sensitivity

Non-celiac gluten sensitivity (NCGS) is described as a condition of multiple symptoms that improves when switching to a gluten-free diet, after celiac disease and wheat allergy are excluded. Recognized since 2010, it is included among gluten-related disorders. Its pathogenesis is not yet well understood, but the activation of the innate immune system, the direct negative effects of gluten and probably other wheat components, are implicated.

NCGS is the most common syndrome of gluten intolerance, with a prevalence estimated to be 6-10%. NCGS is becoming a more common diagnosis, but its true prevalence is difficult to determine because many people self-diagnose and start a gluten-free diet, without having previously tested for celiac disease or having the dietary prescription from a physician. People with NCGS and gastrointestinal symptoms remain habitually in a “no man's land”, without being recognized by the specialists and lacking the adequate medical care and treatment. Most of these people have a long history of health complaints and unsuccessful consultations with numerous physicians, trying to get a diagnosis of celiac disease, but they are only labeled as irritable bowel syndrome. A consistent although undefined number of people eliminate gluten because they identify it as responsible for their symptoms and these improve with the gluten-free diet, so they self-diagnose as NCGS.

People with NCGS may develop gastrointestinal symptoms, which resemble those of irritable bowel syndrome or wheat allergy, or a wide variety of non-gastrointestinal symptoms, such as headache, chronic fatigue, fibromyalgia, atopic diseases, allergies, neurological diseases, or psychiatric disorders, among others. Studies suggest that NCGS may be a chronic disorder, as is the case with celiac disease. Details of NCGS can be found, e.g., in Volta, U., et al. “Nonceliac wheat sensitivity: an immune-mediated condition with systemic manifestations.” Gastroenterology Clinics 48.1 (2019): 165-182, which is incorporated herein by reference in its entirety.

### Wheat Allergy, Gluten Ataxia, and Other Neurological Disorders

People can also experience adverse effects of wheat as result of a wheat allergy. As with most allergies, a wheat allergy causes the immune system to respond abnormally to a component of wheat that it treats as a threatening foreign body. This immune response is often time-limited and does not cause lasting harm to body tissues. Wheat allergy and celiac disease are different disorders. Gastrointestinal symptoms of wheat allergy are similar to those of celiac disease and non-celiac gluten sensitivity, but there is a different interval between exposure to wheat and onset of symptoms. An allergic reaction to wheat has a fast onset (from minutes to hours) after the consumption of food containing wheat and could include anaphylaxis.

Gluten ataxia is an autoimmune disease triggered by the ingestion of gluten. With gluten ataxia, damage takes place in the cerebellum, the balance center of the brain that controls coordination and complex movements like walking, speaking and swallowing, with loss of Purkinje cells. People with gluten ataxia usually present gait abnormality or incoordination and tremor of the upper limbs. Gaze-evoked nystagmus and other ocular signs of cerebellar dysfunction are common. Myoclonus, palatal tremor, and opsoclonus-myoclonus may also appear. Early diagnosis and treatment with a gluten-free diet can improve ataxia and prevent its progression. The effectiveness of the treatment depends on the elapsed time from the onset of the ataxia until diagnosis, because the death of neurons in the cerebellum as a result of gluten exposure is irreversible. Gluten ataxia accounts for 40% of ataxias of unknown origin and 15% of all ataxias. Less than 10% of people with gluten ataxia present any gastrointestinal symptom, yet about 40% have intestinal damage.

In addition to gluten ataxia, gluten sensitivity can cause a wide spectrum of neurological disorders, which develop with or without the presence of digestive symptoms or intestinal damage. These include peripheral neuropathy, epilepsy, headache, encephalopathy, vascular dementia, and various movement disorders (restless legs syndrome, chorea, parkinsonism, Tourette syndrome, palatal tremor, myoclonus, dystonia, opsoclonus myoclonus syndrome, paroxysms, dyskinesia, myorhythmia, myokymia). The diagnosis of underlying gluten sensitivity is complicated and delayed when there are no digestive symptoms. People who do experience gastrointestinal problems are more likely to receive a correct diagnosis and treatment. A strict gluten-free diet is the first-line treatment, which should be started as soon as possible. It is effective in most of these disorders. When dementia has progressed to an advanced degree, the diet has no beneficial effect. Cortical myoclonus appears to be treatment-resistant on both gluten-free diet and immunosuppression.

### Colostrum

Colostrum is the earliest milk produced from the mammary glands for the first few days after giving birth and is unique in its composition of essential nutrients, immune factors, and oligosaccharides that benefit the newborn. In the case of cows, bovine colostrum is produced immediately after calving and quickly wanes to mature milk, which lacks the high level of beneficial nutrients found in bovine colostrum. There are several factors affecting the composition and physical properties of colostrum such as individuality, breed, parity, pre-partum nutrition, length of the dry period of cows, and time post-partum. Generally, colostrum has more fat, protein, peptides, non-protein nitrogen, ash, vitamins and minerals, hormones, growth factors, cytokines, nucleotides, and less lactose compared to mature milk content. The concentration of these compounds decreases rapidly in the first few days (e.g., 1, 2, 3, 4, 5, 6, 7, 8, 9, or 10 days) of lactation with the exception of lactose content.

Colostrum is a complex biological fluid and contains significant components which are natural anti-microbial factors for stimulating the maturation of calf immunity. In addition, the development and function of the GI tract are shaped by colostrum intake, and it also affects the metabolic and endocrine systems as well as the nutritional state of neonatal calves. Colostrum has muscular-skeletal repair and growth potential in addition to its immune support function and many benefits to health because of its content of bioactive proteins. Further, some evidence suggests that the cytokines, immunoglobulins, growth factors, antimicrobial compounds, and maternal immune cells are transferred to the newborn with the feeding of colostrum to support neonatal immunity. Bovine colostrum has even been purported to treat viral and bacterial infections as a nutraceutical. Together, the existing evidence in support of colostrum suggests that there is potential for colostrum to have a significant role in supporting human health as well.

Details of colostrum and its function can be found, e.g., in Arslan, A., et al. “Bovine colostrum and Its potential for human health and nutrition.” Frontiers in Nutrition (2021): 350, which is incorporated herein by reference in its entirety.

In some embodiments, the colostrum described herein includes antibodies (e.g., immunoglobulins) that specifically bind to gluten or partially digested gluten described herein. In some embodiments, the antibody in the composition is homogeneous. In some embodiments, the antibody in the composition is non-homogeneous. In some embodiments, the antibody in the composition is polyclonal antibodies (e.g., antibodies that are secreted by different B cell lineages). In some embodiments, the antibody specifically binds to PT-gliadin.

### Generation of Gluten-Specific Antibodies in Colostrum

In some embodiments, provided herein are methods of immunizing an animal for production of colostrum, which includes antibodies (e.g., immunoglobulins) that specifically bind to gluten or partially digested gluten. In some embodiments, the methods include administering (e.g., injecting) the gluten or partially digested gluten (e.g., PT-gliadin) to the animal, optionally with an adjuvant; and collecting one or more milkings from the animal after parturition. In some embodiments, at least one of the one or more milkings includes the colostrum described herein.

In some embodiments, the animal is a non-human mammal, e.g., a cow, buffalo, goat, reindeer, horse, sheep, camel, yak, or any animal whose colostrum or milk product can be orally administered to human safely, preferably without inducing significant immune response.

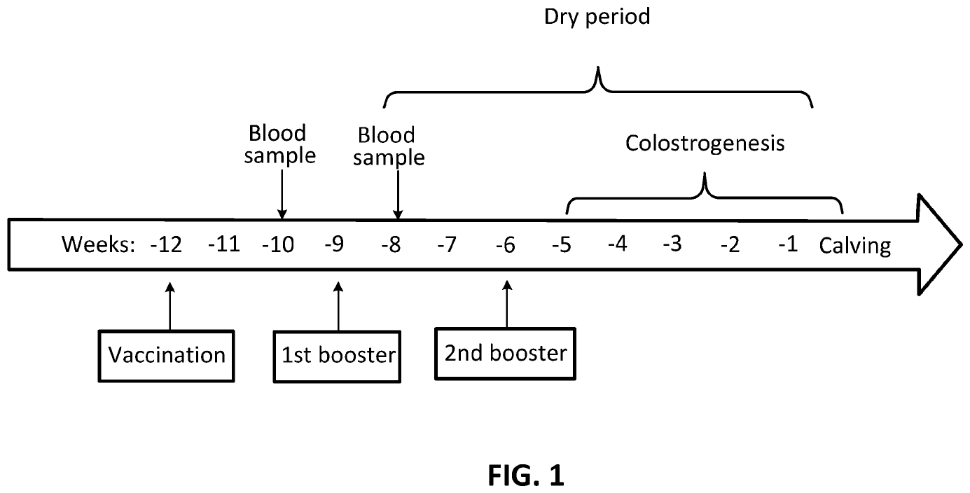

In some embodiments, the animal is pregnant when immunized (e.g., injected) with gluten or partially digested gluten (e.g., PT-gliadin), such that the animal can generate antibodies (e.g., immunoglobulins) that specifically bind to gluten or partially digested gluten. In some embodiments, the pregnant animal is immunized (e.g., injected) at least once, at least twice, at least three times, at least four times, at least five times, or at least six times prior to parturition. In some embodiments, the interval between each injection is at least 1 week, at least 2 weeks, at least 3 weeks, or at least 4 weeks. In some embodiments, the pregnant animal (e.g., cow) is immunized before and during the dry period (e.g., from about 8 weeks to parturition). In some embodiments, the pregnant animal (e.g., cow) is immunized before the dry period. In some embodiments, the pregnant animal (e.g., cow) is immunized prior to colostrogenesis (i.e., the prepartum transfer of immunoglobulins from maternal circulation into mammary secretions). In some embodiments, the pregnant animal (e.g., cow) is immunized at about 15 weeks, about 14 weeks, about 13 weeks, about 12 weeks, about 11 weeks, about 10 weeks, about 9 weeks, about 8 weeks, about 7 weeks, about 6 weeks, or about 5 weeks prior to parturition (e.g., calving). For example, a pregnant cow can be immunized at about 12 weeks (e.g., from about 14 weeks to about 10 weeks, from about 14 weeks to about 11 weeks, from about 14 weeks to about 12 weeks from about 14 weeks to about 13 weeks, from about 13 weeks to about 10 weeks, from about 13 weeks to about 11 weeks, from about 13 weeks to about 12 weeks, from about 12 weeks to about 10 weeks, from about 12 weeks to about 11 weeks, or from about 11 weeks to about 10 weeks), at about 9 weeks (e.g., from about 10 weeks to about 7 weeks, from about 10 weeks to about 8 weeks, from about 10 weeks to about 9 weeks, from about 9 weeks to about 7 weeks, from about 9 weeks to about 8 weeks, or from about 8 weeks to about 7 weeks), and/or at about 6 weeks (e.g., from about 7 weeks to about 5 weeks, from about 7 weeks to about 6 weeks, or from about 6 weeks to about 5 weeks) prior to calving. In some embodiments, the immunization schedule is described in FIG. 1.

In some embodiments, the animal is immunized according to the animal's gestation length. For example, cows have a gestation length of about 279-287 days, and one or more immunizations are carried out within the about 15 weeks to about 5 weeks prior to calving. In some embodiments, the animal is immunized one or more times at the late phase of gestation, e.g., within about 40%, about 35%, about 30%, about 25%, about 20%, about 15%, or about 10% of the animal's gestation length prior to parturition.

In some embodiments, one or more milkings are collected from the animal after parturition. For example, the first milking can be collected within about 1 hour, about 2 hours, about 3 hours, about 4 hours, about 5 hours, about 6 hours, about 7 hours, about 8 hours, about 9 hours, about 10 hours, about 11 hours, or about 12 hours after parturition. In some embodiments, at least 1, 2, 3, 4, 5, 6, 7, or 8 milkings are collected. In some embodiments, the interval between each milking is about 8 hours, about 9 hours, about 10 hours, about 11 hours, about 12 hours, about 13 hours, about 14 hours, about 15 hours, or about 16 hours. In some embodiments, a total of about 2-4 liters (e.g., about 2-3.5 liters, about 2-3 liters, about 2-2.5 liters, about 2.5-4 liters, about 2.5-3.5 liters, about 2.5-3 liters, about 3-4 liters, about 3-3.5 liters, or about 3.5-4 liters of product are collected per milking. In some embodiments, the gluten-specific antibodies described herein exist in the first, the second, the third, the fourth, the fifth, the sixth, the seventh, and/or the eighth milkings. In some embodiments, the milkings are collected manually or with automated devices.

In some embodiments, the colostrum generated using the methods described herein (e.g., by immunizing pregnant cows) has a concentration of gluten-specific antibodies that is at least 1-fold, 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, 100-fold, 500-fold, 1000-fold, 5000-fold, or 10000-fold higher than that in a non-colostrum milk sample, that of a commercial colostrum (e.g., Colostrum 200), or that of a reference colostrum sample that is produced by an animal without being immunized by the gluten or partially digested gluten.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, antibody fragments, and multi-specific antibodies formed from antibody fragments. As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′) 2, and Fv fragments. In some embodiments, the antibody is an immunoglobulin.

As used herein, the term “gluten-specific antibody” refers to an antibody or antigen-binding fragment thereof that specifically binds to gluten or partially digested gluten.

In some embodiments, the colostrum described herein can be processed using the methods described herein, e.g., skimming, decaseination, heating, and/or pasteurization. In some embodiments, the processed colostrum has a concentration of gluten-specific antibodies of at least 1 mg/mL, at least 2 mg/mL, at least 3 mg/mL, at least 4 mg/mL, at least 5 mg/mL, at least 6 mg/mL, at least 7 mg/mL, at least 8 mg/mL, at least 9 mg/mL, at least 10 mg/mL, at least 15 mg/mL, at least 20 mg/mL, at least 25 mg/mL, at least 30 mg/mL, at least 35 mg/mL, at least 40 mg/mL, at least 45 mg/mL, or at least 50 mg/mL. In some embodiments, the concentration is at least 7.5 mg/mL, at least 7.6 mg/mL, at least 7.7 mg/mL, at least 7.8 mg/mL, at least 7.9 mg/mL, at least 8 mg/mL, at least 8.1 mg/mL, at least 8.2 mg/mL, at least 8.3 mg/mL, at least 8.4 mg/mL, or at least 8.5 mg/mL. In some embodiments, the concentration is about 1-50 mg/mL, about 1-40 mg/mL, about 1-30 mg/mL, about 1-20 mg/mL, about 1-10 mg/mL, about 1-5 mg/mL, about 5-50 mg/mL, about 5-40 mg/mL, about 5-30 mg/mL, about 5-20 mg/mL, about 5-10 mg/mL, about 10-50 mg/mL, about 10-40 mg/mL, about 10-30 mg/mL, about 10-20 mg/mL, about 20-50 mg/mL, about 20-40 mg/mL, about 20-30 mg/mL, about 30-50 mg/mL, about 30-40 mg/mL, or about 40-50 mg/mL. In some embodiments, the concentration is about 1-5 mg/mL, about 5-10 mg/mL, about 10-15 mg/mL, about 15-20 mg/mL, about 20-25 mg/mL, about 25-30 mg/mL, about 30-35 mg/mL, about 35-40 mg/mL, about 40-45 mg/mL, or about 45-50 mg/mL.

In some embodiments, the animal (e.g., cow) already has 1, 2, 3, 4, 5, 6, 7, 8, 9, or 10 prior gestations prior to pregnancy.

In some embodiments, the partially digested gluten (e.g., PT-gliadin) used for immunizing the animals can be produced according to methods described in Junker, Y., et al. “Wheat amylase trypsin inhibitors drive intestinal inflammation via activation of toll-like receptor 4.” Journal of Experimental Medicine 209.13 (2012): 2395-2408, which is incorporated herein by reference in its entirety. In some embodiments, the dose level of the partially digested gluten (e.g., PT-gliadin) is about 40-80 mg (e.g., 58 mg) per cow. In some embodiments, the dose level is about 0.05 mg/kg, about 0.06 mg/kg, about 0.07 mg/kg, about 0.08 mg/kg, about 0.09 mg/kg, about 0.1 mg/kg, about 0.11 mg/kg, about 0.12 mg/kg, about 0.13 mg/kg, about 0.14 mg/kg, or about 0.15 mg/kg. In some embodiments, the dose level is about 0.01-0.5 mg/kg, about 0.05-0.4 mg/kg, about 0.05-0.3 mg/kg, about 0.05-0.2 mg/kg, or about 0.05-0.15 mg/kg.

### Method of Alleviating Symptoms or Treatment

The compositions described herein can be used for various therapeutic purposes.

In one aspect, the disclosure provides methods for treating a gluten-related disorder in a subject, or methods of alleviating symptoms of a gluten-related disease in a subject. In some embodiments, the antibodies (e.g., immunoglobulins) that specifically bind to gluten or partially digested gluten in colostrum can inhibit and/or interfere absorption of the gluten or partially digested gluten in GI tract of the subject (e.g., human). In some embodiments, the methods can result in reduction of the severity and/or duration of one or more symptoms of the gluten-related disorder (e.g., celiac disease) in a subject. For example, a subject (e.g., a human with celiac disease) can consume the colostrum or a composition including the colostrum described herein immediately before starting a meal. In some embodiments, the colostrum, processed colostrum, or a composition including the colostrum described herein can be orally consumed within about 2 hours, about 1.5 hours, about 1 hour, about 50 minutes, about 40 minutes, about 30 minutes, about 20 minutes, about 19 minutes, about 18 minutes, about 17 minutes, about 16 minutes, about 15 minutes, about 14 minutes, about 13 minutes, about 12 minutes, about 11 minutes, about 10 minutes, about 9 minutes, about 8 minutes, about 7 minutes, about 6 minutes, about 5 minutes, about 4 minutes, about 3 minutes, about 2 minutes, about 1 minutes, about 50 seconds, about 40 seconds, about 30 seconds, about 20 seconds, about 10 seconds, about 5 seconds before having a meal. In some embodiments, the colostrum, processed colostrum, or a composition including the colostrum described herein can be orally consumed by a subject at substantially the same time of having a meal. In some embodiments, one or more drugs to protect antibodies (e.g., immunoglobulins) in the colostrum and/or to reduce the amount of the antibodies being digested in the GI tract (e.g., stomach) is also consumed by the subject. In some embodiments, the one or more drugs include a proton pump inhibitor (e.g., omeprazole). In some embodiments, the one or more drugs include omeprazole (e.g., about 10 mg˜about 30 mg, about 15 mg˜about 25 mg, or about 25 mg).

In one aspect, the disclosure features methods that include administering a therapeutically effective amount of the colostrum or the processed colostrum including gluten-specific antibodies, or the composition including the colostrum described herein to a subject in need thereof, e.g., a subject having a gluten-related disorder. In some embodiments, the gluten-related disorder is celiac disease, non-celiac gluten sensitivity (NCGS), or gluten intolerance. In some embodiments, the gluten-related disease is any one of the gluten-related diseases described herein.

In some embodiments, the methods described herein can be used to alleviate, reduce, or improve symptoms of celiac disease. In some embodiments, the methods described herein can be used to reduce gluten sensitivity or reduce non-celiac gluten sensitivity. In some embodiments, the methods described herein can be used to treat celiac disease.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, by an “effective amount” is meant an amount or dosage sufficient to effect beneficial or desired results including halting, slowing, retarding, or inhibiting progression or relieving symptoms of a disease, e.g., a gluten-related disorder. An effective amount will vary depending upon, e.g., an age and a body weight of a subject to which the colostrum, the gluten-specific antibodies, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment is an amount sufficient to ameliorate, stop, stabilize, reverse, inhibit, slow and/or delay progression of a gluten-related disorder in a patient or is an amount sufficient to alleviate, ameliorate, stop, stabilize, reverse, slow and/or delay symptoms of the disease. As is understood in the art, an effective amount of an antibody or antigen binding fragment may vary, depending on, inter alia, patient history as well as other factors such as the type (and/or dosage) of antibody used.

Effective amounts and schedules for administering the antibodies, antibody-encoding polynucleotides, and/or compositions disclosed herein may be determined empirically, and making such determinations is within the skill in the art. Those skilled in the art will understand that the dosage that must be administered will vary depending on, for example, the subject (e.g., human) that will receive the colostrum, the gluten-specific antibodies, and/or compositions thereof disclosed herein, the route of administration, the particular type of the colostrum, antibodies, antigen binding fragments, and/or compositions disclosed herein used and other drugs being administered to the subject.

A typical daily dosage of an effective amount of an antibody is 0.01 mg/kg to 100 mg/kg (mg per kg of patient weight). In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4.5 mg/kg, 4 mg/kg, 3.5 mg/kg, 3 mg/kg, 2.5 mg/kg, 2.4 mg/kg, 2.3 mg/kg, 2.2 mg/kg, 2.1 mg/kg, 2 mg/kg, 1.9 mg/kg, 1.8 mg/kg, 1.7 mg/kg, 1.6 mg/kg, 1.5 mg/kg, 1.4 mg/kg, 1.3 mg/kg, 1.2 mg/kg, 1.1 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg. In some embodiments, the dosage is about 0.1-10 mg/kg, about 0.1-5 mg/kg, about 0.1-2 mg/kg, about 0.1-1 mg/kg, about 0.5-10 mg/kg, about 0.5-5 mg/kg, about 0.5-2 mg/kg, or about 1-2 mg/kg. In some embodiments, the dosage is about 0.1-0.5 mg/kg, about 0.5-1 mg/kg, about 1-1.5 mg/kg, about 1.5-2 mg/kg, about 2-2.5 mg/kg, about 2.5-5 mg/kg, or about 5-10 mg/kg.

In some embodiments, about 1 g˜about 100 g, about 1 g˜about 50 g, about 1 g˜ about 40 g, about 10 g˜about 100 g, about 10 g˜about 50 g, about 20 g˜about 50 g, about 10 g˜about 20 g, about 15 g˜about 20 g, about 15 g˜about 30 g of the colostrum is administered to a subject (e.g., an adult or a juvenile). In some embodiments, at least 5, 10, 15, 20, 25, 30, 35, 40, 45, or 50 g of the colostrum is administered to the subject.

In any of the methods described herein, the colostrum, the gluten-specific antibodies, and/or compositions thereof (e.g., any of the colostrum, the gluten-specific antibodies, and/or compositions thereof described herein) and, optionally, at least one additional therapeutic agent can be co-administered to the subject, immediately prior to having a meal. In some embodiments, at least two different types of colostrum, the gluten-specific antibodies, and/or compositions thereof are administered in the same composition (e.g., a liquid composition). In some embodiments, at least one type of colostrum, the gluten-specific antibodies, and/or compositions thereof and at least one additional therapeutic agent are administered in the same composition (e.g., a liquid composition). In some embodiments, at least one type of colostrum, the gluten-specific antibodies, and/or compositions thereof and the at least one additional therapeutic agent are administered in two different compositions (e.g., a liquid composition containing at least one type of colostrum, the gluten-specific antibodies, and/or compositions thereof and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation. In some embodiments, the at least one additional therapeutic agent is a drug to prevent degradation of colostrum in the GI tract, e.g., by stomach acidity. In some embodiments, the at least one additional therapeutic agent is omeprazole.

In some embodiments, the one or more additional therapeutic agents can be administered to the subject prior to, or after administering the at least one type of colostrum, the gluten-specific antibodies, and/or compositions thereof (e.g., any of the colostrum, the gluten-specific antibodies, and/or compositions thereof described herein). In some embodiments, the one or more additional therapeutic agents and the at least one type of colostrum, the gluten-specific antibodies, and/or compositions thereof (e.g., any of the colostrum, the gluten-specific antibodies, and/or compositions thereof described herein) are administered to the subject such that there is an overlap in the bioactive period of the one or more additional therapeutic agents and the colostrum, the gluten-specific antibodies, and/or compositions thereof (e.g., any of the colostrum, the gluten-specific antibodies, and/or compositions thereof described herein) in the subject.

In some embodiments, the subject can be administered the at least one type of colostrum, the gluten-specific antibodies, and/or compositions thereof (e.g., any of the colostrum, the gluten-specific antibodies, and/or compositions thereof described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled person in the art may determine the length of the treatment period using any of the methods described herein for diagnosing or following the effectiveness of treatment (e.g., the observation of at least one symptom of gluten-related disorder). As described herein, a skilled person in the art can also change the identity and number (e.g., increase or decrease) of the colostrum, the gluten-specific antibodies, and/or compositions thereof (and/or one or more additional therapeutic agents) administered to the subject and can also adjust (e.g., increase or decrease) the dosage or frequency of administration of the colostrum, the gluten-specific antibodies, and/or compositions thereof (and/or one or more additional therapeutic agents) to the subject based on an assessment of the effectiveness of the treatment (e.g., using any of the methods described herein and known in the art). In some embodiments, the colostrum, the gluten-specific antibodies, and/or compositions thereof described herein can be used as medicinal food.

In some embodiments, the disclosure is related to a lyophilized composition including the colostrum described herein. For example, the colostrum or the composition including the colostrum described herein can be processed using the methods described herein and finally freeze-dried, e.g., to make the colostrum more palatable. The methods include removing fat and/or casein to get a product with higher concentration of immunoglobulins; pasteurization; and/or heating the product at about 55-65° C. (e.g., 60° C.) for about 0.5-2 hours (e.g., 0.5 hour, 1 hour, 1.5 hours, or 2 hours). In some embodiments, the processed colostrum or lyophilized composition thereof (e.g., in the form of powder) does not substantially reduce the percentage of gluten-specific antibodies (e.g., immunoglobulins) in the product. For example, at least 80%, at least 85%, at least 90%, at least 95%, at least 96%, at least 97%, at least 98%, or at least 99% of the gluten-specific antibodies (e.g., immunoglobulins) are preserved after processing and/or freeze-dry.

### EXAMPLES

The invention is further described in the following examples, which do not limit the scope of the invention described in the claims.

### Example 1. Generation of PT-Gliadin

Pepsin-trypsin resistant gliadin (PT-gliadin) was produced in laboratory according to methods described in Junker, Y., et al. “Wheat amylase trypsin inhibitors drive intestinal inflammation via activation of toll-like receptor 4.” Journal of Experimental Medicine 209.13 (2012): 2395-2408. Specifically, 5 grams of gliadin (Sigma Aldrich) in 50 mL was digested with 25 mg pepsin in 0.1 M HCl, pH 1.8, at 37° C. for 4 hours (substrate/enzyme ratio of 200:1). Afterwards, pH was adjusted to 7.8 and the pepsin-digested gliadin was further digested with 25 mg trypsin at 37° C. for 4 hours (substrate/enzyme ratio of 200:1). The pH was then adjusted to 4.5, resulting in a precipitate which was removed by centrifugation at 2,500 rpm. The product was boiled for 30 minutes to inactivate both pepsin and trypsin (100° C.). Supernatant containing the PT-gliadin was dialyzed against 10 mM ammonium carbonate, pH 7.8. The product was sterilized by filtering through 0.2 micron filters. Finally, the product was freeze-dried (48 hours, −80° C.) and a limulus amebocyte lysate (LAL) assay was performed to confirm the absence of toxic levels of bacterial endotoxin for cows (Pyrogent Plus Gel Clot LAL).

### Example 2. Preparation of Vaccines

250 μg of gliadin was effective in stimulating immunity in hens (Gujral, N., et al. “Effect of anti-gliadin IgY antibody on epithelial intestinal integrity and inflammatory response induced by gliadin.” BMC Immunology 16.1 (2015): 1-11; and Stadlmann, V., et al. “Novel avian single-chain fragment variable (scFv) targets dietary gluten and related natural grain prolamins, toxic entities of celiac disease.” BMC Biotechnology 15.1 (2015): 1-14). Acknowledging that hens weigh about 2.5 kg while cows are about 580 kg, the optimal dose of gliadin would be around 58 mg for cows. This dose level is in line with previous studies that administered 50 mg of Clostridium difficile toxins to cows with a good immune response (Kelly, C. P., et al. “Anti-Clostridium difficile bovine immunoglobulin concentrate inhibits cytotoxicity and enterotoxicity of C. difficile toxins.” Antimicrobial Agents and Chemotherapy 40.2 (1996): 373-379).

Moreover, as PT-gliadin is soluble in PBS at a concentration of 1 mg/mL, to maintain an appropriate volume for the injections, 5 mg PT-gliadin in 5 mL of PBS+5 mL of incomplete Freund Adjuvant were co-administered per injection. Thus, a total of 50 mg PT-gliadin in 10 injections were administered per immunization.

### Example 3. Cows and Immunization Schedule

10 pregnant cows were selected, and each cow already had 3-4 prior gestations. The cohort included 8 black Holstein, 1 brown Holstein and 1 Jersey. The cows were vaccinated according to the schedule outlined in FIG. 1. For maximum antibody transfer into the mammary gland, there is usually a peak in serum antibodies concentration at the time of the colostrogenesis period, which starts around 5 weeks prior to calving. Therefore, to maximise the immune response, the cows were first immunized at 12 weeks prior to calving, followed by 2 boosters at 9 weeks and 6 weeks prior to calving.

As shown in FIG. 1, 10 cows were immunized at the late phase of their gestation. The immunizations were well tolerated, with minor side effects (induration at the site of injection (n=2) and fever on the day after immunization (n=1)).

All cows calved live infants except for one stillborn (No. 1363). There was one twin pregnancy (No. 1328).

### Example 4. ELISA Testing on Blood Samples Post Vaccination

An ELISA assay was developed for identifying and quantifying anti-PT-gliadin IgG in bovine serum using PT-gliadin at a concentration of 0.5 mg/50 μL and a blocking agent with 1% Donor Horse Serum PBS-T. Briefly, 96-well ELISA plates were coated with 0.5 mg of PT-gliadin per well overnight at 4° C. Blocking agent (1% Donor Horse Serum in PBS-0.05% Tween® 20 (PBS-T)) was added and the plates were incubated for 60 minutes at room temperature. After the incubation, the plates were washed. The serum samples were diluted at ratios of 1:500, 1:1000, 1:2000, and 1:4000 using Tris-Buffered Saline. 100 μL of the serial dilution of serum IgG were added to the wells and incubated for 60 minutes at room temperature. After the incubation, the plates were washed again and 100 μL of HRP-rabbit anti-bovine IgG (1:1200) in PBS-T was added to each well and the plates were incubated for 60 minutes at room temperature. After three washes with PBS-T, the color reaction was developed using TMB Peroxidase substrate and Peroxidase Substrate solution. After developing for 5 minutes, the reaction was stopped by adding KLP TMB Stop Solution. Optical density (OD) was read at 450 nm (OD450).

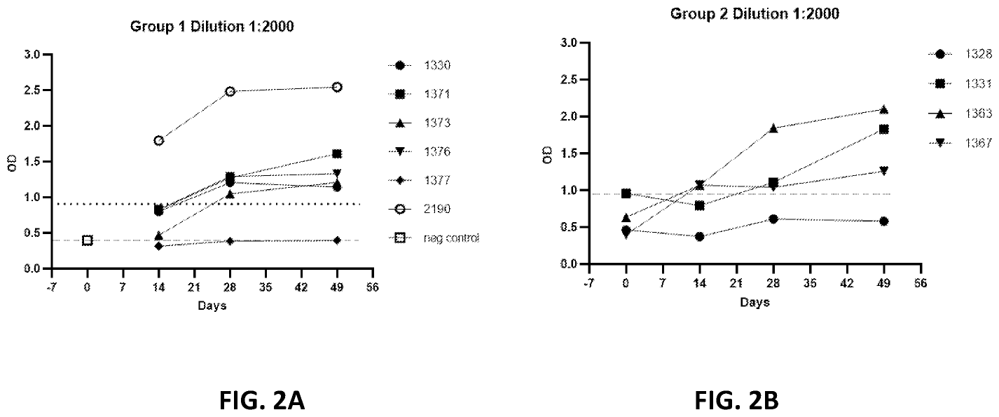

As shown in FIGS. 2A-2B, the results show that the vaccine induced production of gliadin-specific antibodies in the majority of cows. Specifically, eight cows had a significant rise in their OD for anti-PT-gliadin IgG. Cow 2190, who generated significantly higher amounts of antibodies, was the only Jersey in 10 experimental cows. Two cows (No. 1328 and No. 1377) did not produce antibodies. It is likely that Jerseys can produce more concentrated milk than Holsteins, therefore generating higher amount of antibodies.

### Example 5. ELISA Testing on Colostrum Samples Post Vaccination

After colostrum was produced, an ELISA assay was developed to identify anti-PT-gliadin IgG in the colostrum, using PT-gliadin as the capture antigen at a concentration of 0.5 mg/50 μL, as well as a blocking agent with 0.05% solution of gelatin from cold water fish skin in PBS-T. Briefly, colostrum was diluted using tris-buffered saline-Tween® 20 (TBS-T) with final dilution factors of 1×10⁴, 5×10⁴, 1×10⁵, and 5×10⁵. Dilution series were created in separate test tubes and final solutions were transferred to individual wells at the appropriate time in the ELISA protocol. All dilutions were duplicated so that a total of 12 wells (3 wells per dilution factor) contained solutions representing the same colostrum sample. Wells were coated with 50 μL of PT-gliadin (0.5 mg/50 μL). A 0.05% solution of gelatin from cold water fish skin in PBS-T (Cat #: G7041; Sigma-Aldrich Inc., St. Louis, MO) was used as a blocking agent and 200 μL were added to each well. 100 μL sample was added in each well and incubated at room temperature for 60 minutes. After 3 washes with PBS-T, 100 μL of HRP-rabbit anti-bovine IgG diluted at 1:1200 in PBS-T was added. After another three washes with PBS-T, a color reaction was developed using TMB Peroxidase substrate and Peroxidase Substrate solution. After developing for 5 minutes, the reaction was stopped by adding KLP TMB Stop Solution. Optical density (OD) was read at 450 nm (OD450).

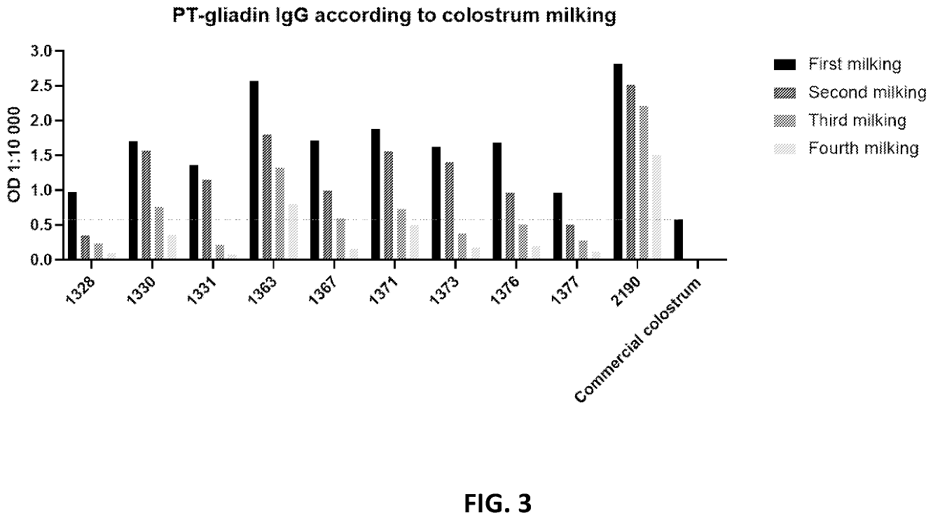

First four milkings were collected. The first milking was collected as soon as possible after parturition. Volumes were variable from one cow to another, but everything that could be collected with the automated device was. Subsequent milkings were collected at 12-hour intervals. A total of 2-4 liters of product were obtained per milking. The calves received commercial colostrum. As shown in FIG. 3, the results show that PT-gliadin antibodies were identified in the colostrum (significantly higher than Colostrum 200 (SSCL, Canada)), with their concentrations being the highest in the first milking and rapidly declining afterwards.

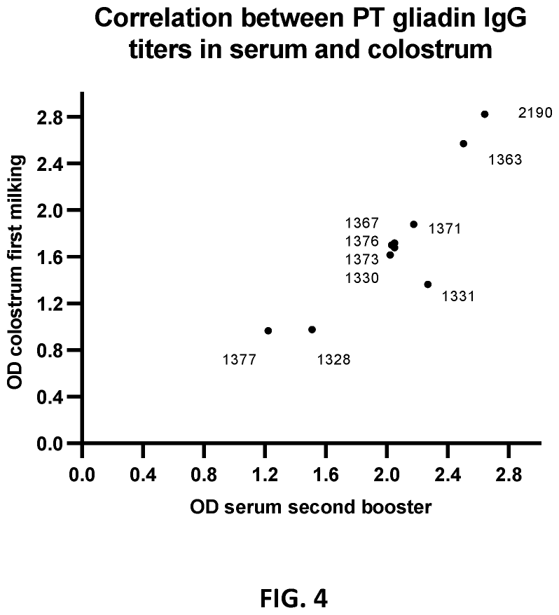

Notably, a good correlation was observed between the pre-calving serum titers and colostrum titers. As shown in FIG. 4, the two cows who did not show a rise in their circulating antibodies did not have a significant amount of antibodies in the colostrum (1328 and 1377).

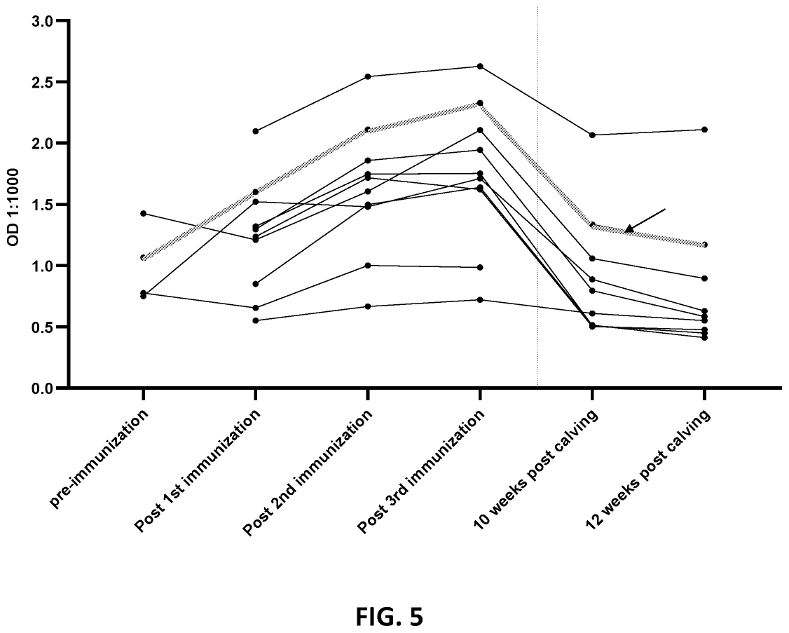

The mature milk produced by the cows were also tested by ELISA, and no PT-gliadin antibodies were detected. It is contemplated that transfer of immunoglobulins from blood to milk in pregnant cows may only occur in the few weeks before parturition. Furthermore, as shown in FIG. 5, the level of circulating antibodies significant decreased in all cows after calving. In addition, there was no transfer of the antibodies to the calves, except for two who potentially drank some colostrum before being separated from their mother cows (MW2 and MW3).

### Example 6. Processing Colostrum for Human Consumption

To make colostrum more palatable and to remove fat and casein from colostrum such that the product has a higher concentration of immunoglobulins (hyperimmune colostrum), industry standards to skim and decaseinate the raw colostrum were followed. The final product of colostrum was then proceeded to freeze-dry.

The colostrum was processed using the method as follows. The colostrum was skimmed by centrifugation at 9000 g for 30 minutes at 4° C. The supernatant (containing fat) was removed and the skimmed colostrum was retained. Rennet (New England Cheese making Supply) at a concentration of 2.5 mL per 3784 mL of colostrum was used for decaseination. Specifically, the colostrum was mixed with rennet on a magnetic stirrer at 270/min 35° C. for 30 minutes. The product was then centrifuged at 4200 g for 30 minutes at 25° C. to separate only the clear lactoserum, which was then heated at 60° C. for 60 minutes, and finally freeze-dried. The final product was tested by a food safety laboratory to confirm the absence of microbial contamination, solvents and pesticides.

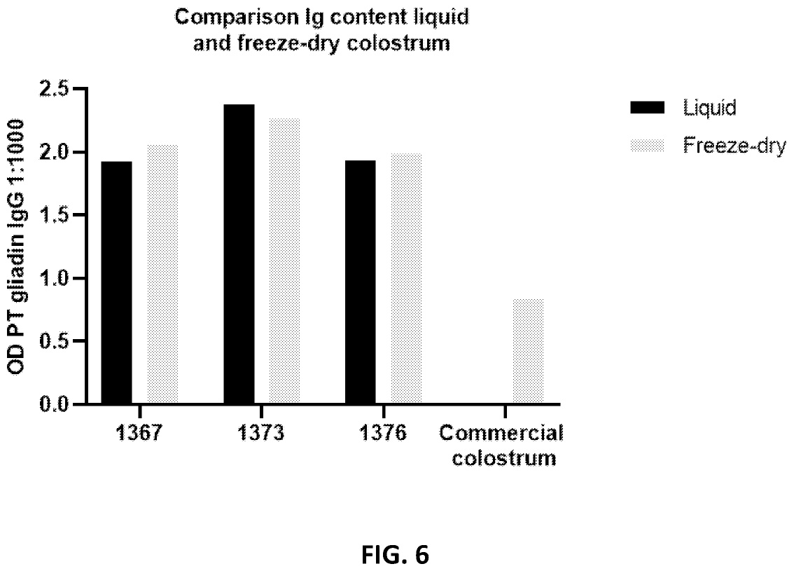

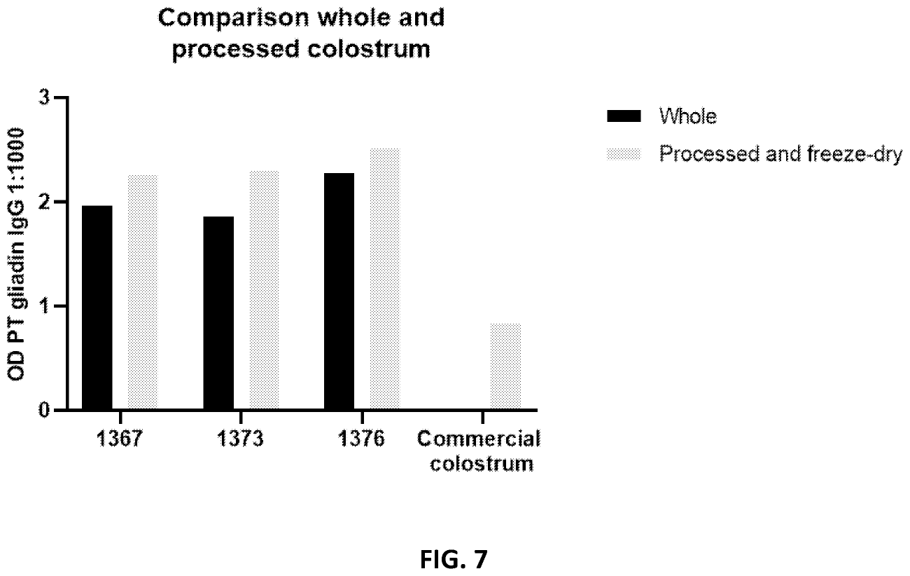

As shown in FIG. 6, the results demonstrate that freeze-dryer did not alter the immunoglobulin composition of the colostrum. As shown in FIG. 7, the processed colostrum showed an increase in the concentration of immunoglobulins after skimming and decaseination.

Subsequent experiments were also performed using the processed colostrum. According to standard scales and kit detecting general IgG (Bethyl Laboratories Inc., LuBioScience, Luzern, Switzerland), it was estimated that every mL of processed colostrum contained about 8.1 mg of PT-gliadin IgG.

### Example 7. Colostrum Binding to Gliadin

A column-based experiment was performed to assess the binding of the colostrum with gliadin. Specifically, PT-gliadin and the processed colostrum were combined at different ratios of 1:1, 1:5 and 1:25 respectively, as shown in the table below, prior to a 10-minute incubation at room temperature for subsequent column analysis.

TABLE 1
Different ratios of gliadin and colostrum used for the experiment.

| Gliadin | Colostrum | Ratio |
|---|---|---|
| 1 | 1 | 1:1 |
| 1 | 5 | 1:5 |
| 1 | 25 | 1:25 |

Protein G columns were prepared and equilibrated per manufacturer instructions (Thermo Scientific). Specifically, 100 μL of the mixture of PT-gliadin and the hyperimmune colostrum was loaded onto a protein G column for an incubation at room temperature with end-over-end mixing for 10 minutes. Subsequently, the unbound gliadin in the collection tubes were identified by a R5 ELISA assay (Rida Screen).

This column experiment involves the utilization of three types of colostrum samples, including two individual colostrum samples with the highest amount of anti-PT-gliadin antibodies, referred to as hyperimmune colostrum #1 and hyperimmune colostrum #2, and a mixed sample made by combining the top three colostrum samples (No. 2190, 1363 and 1371), referred to as the mix of 3 hyperimmune colostrum.

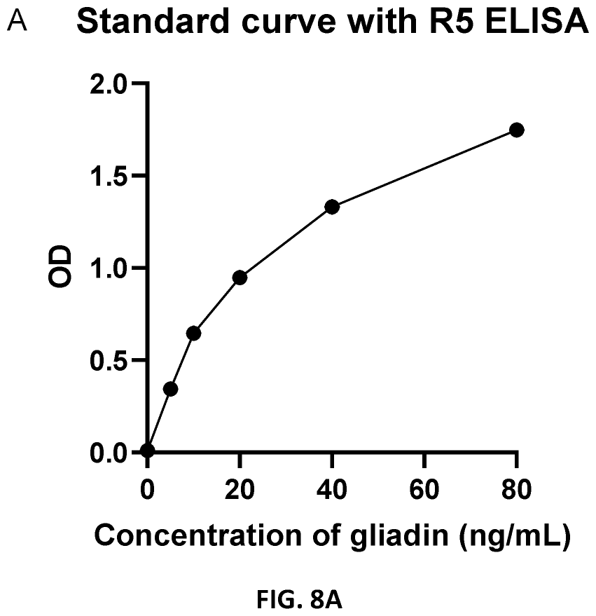

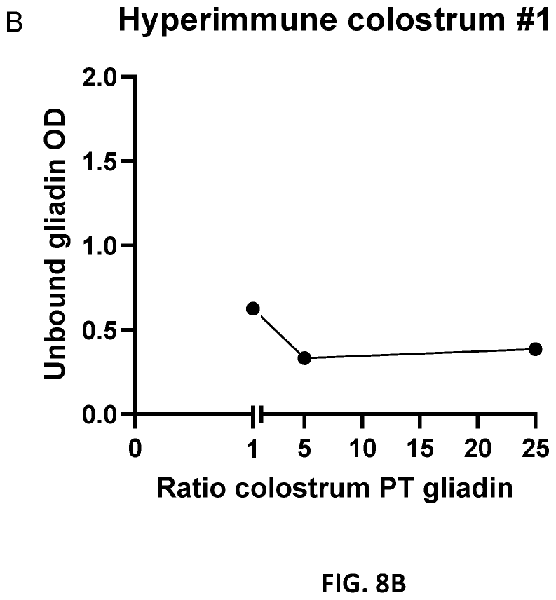

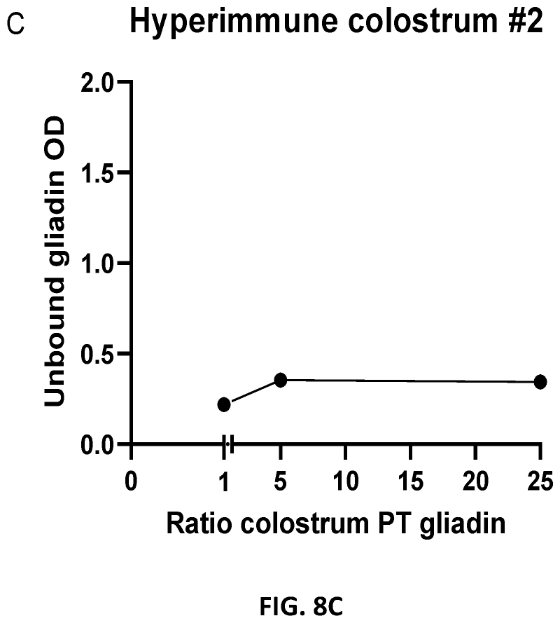

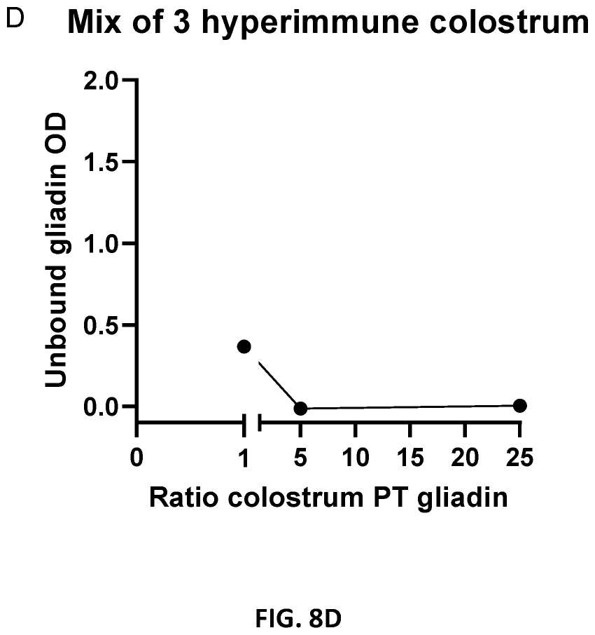

As shown in FIG. 8A, the unbound PT-gliadin exhibited a standard curve of increasing OD values corresponding to the concentration of the loaded PT-gliadin. As shown in FIG. 8B, hyperimmune colostrum #1 demonstrated the evident binding capacity to PT-gliadin, specifically at the ratios of 1:5 and 1:25, resulting in a noticeable reduction in unbound PT-gliadin. In contrast, as shown in FIG. 8C, a consistent unbound PT-gliadin level was observed in the reaction with hyperimmune colostrum #2 at various ratios, indicating the lack of binding interaction between hyperimmune colostrum #2 and PT-gliadin. Notably, FIG. 8D and Table 2 demonstrate that the mix of 3 hyperimmune colostrum exhibited complete binding to PT-gliadin at the ratios of 1:5 and 1:25 where no residual unbound PT-gliadin was detected in the collection tubes.

TABLE 2
Summary of the OD readings for each column experiment presented
based on the ratio of PT-gliadin and various colostrum samples

| | Hyperimmune | Hyperimmune | Mix of 3 hyperimmune |
|---|---|---|---|
| | colostrum #1 | colostrum #2 | colostrum |
| 1:1 | 0.625 | 0.2185 | 0.367 |
| 1:5 | 0.3325 | 0.3535 | −0.013 |
| 1:25 | 0.3865 | 0.3445 | 0.0045 |

### Example 8. Clinical Trial of Bovine Colostrum to Prevent Absorption of Gluten

Clinical trials were designed to investigate the use of hyperimmune bovine colostrum to reduce gluten absorption, alleviate symptoms of celiac disease, reduce gluten sensitivity and/or reduce non-celiac gluten sensitivity. A randomized, double-blind, cross-over, interventional study has been performed in which persons who are following a strict gluten-free diet will be challenged with oral gluten with or without the bovine colostrum. The study involves 5 visits for each participant. At the second and fourth visit, they will be subjected to a dietary supplement intervention, taking gluten either with bovine colostrum (1 g of gluten+18 g of bovine colostrum) or with a placebo (1 g of gluten+18 g of placebo). Follow-up duration will be until three days after the last study visit.

This study includes two arms: experimental group in which subjects will receive bovine colostrum with gluten and placebo group in which subjects will receive a placebo with gluten. In the experimental group (gluten+bovine colostrum), participants will receive 18 g of bovine colostrum with apple sauce and 1 g of gluten. In the placebo group (gluten+placebo), participants will receive 18 g of placebo with apple sauce and 1 g of gluten.

Each participant will be given daily proton pump inhibitor (PPI) therapy (omeprazole 20 mg) at least one week before the first intervention, and for the entire duration of the study. Each participant will be invited to fill a celiac disease patient reported outcomes questionnaire (Celiac Disease Symptoms Diary) for the 24 hours prior the study intervention and three days after. Blood and urine samples will be collected during the 24 hours following each study intervention (gluten+colostrum or gluten+placebo)

Adverse events will be recorded during the entire study from enrollment to termination (termination will occur 3 days after the second challenge at the end of the patient reported outcome (PRO) recording period).

The estimated enrollment is 10 participants, ranging in age from 18 years to 75 years, and including both sexes. The participants can be healthy volunteers or individuals have self-reported strict adherence to a gluten-free diet for at least 3 months. The later may be a lifestyle choice, in solidarity with a child or other family/household member with celiac disease, gluten sensitivity or non-celiac gluten sensitivity. Individuals will be excluded from the study if they meet any of the following criteria:
1) Definite or probable gluten exposure during the 72 hours preceding each study intervention visit
2) Known active gastrointestinal disease.
3) Use of an oral digestive enzyme supplement during the 72 hours preceding each study intervention (challenge) visit.
4) History of severe symptomatic reactions to gluten or milk proteins
5) History of allergy to beef or meat
6) History of allergy to apple
7) Severe lactose intolerance
8) Any medication or medical condition which, in the opinion of the investigators, could adversely affect the patient's participation in the trial.
9) Allergy to PPI or other contraindication for PPI use (i.e history of interstitial nephritis attributed to PPI, history of PPI-induced diarrhea)
10) Pregnant women (according to pregnancy test)

The primary outcome measure will be gluten immunogenic peptides in urine. The intra-subject area under the curve (AUC) for gluten immunogenic peptides will be measured in urine following gluten challenge with placebo compared to the AUC for gluten immunogenic peptides following gluten challenge combined with colostrum. The second outcome measure will be symptom score. Intra-subject maximal CDSD scores within a single domain for the 24 hours following gluten challenge.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising colostrum, wherein the colostrum comprises an immunoglobulin that specifically binds to gluten or partially digested gluten.

2. The composition of claim 1, wherein the colostrum is from a non-human mammal.

3. The composition of claim 1 or 2, wherein the colostrum is from a cow (e.g., black Holstein, brown Holstein, or Jersey), buffalo, goat, reindeer, horse, sheep, camel, or yak.

4. The composition of any one of claims 1-3, wherein the gluten or partially digested gluten is gliadin or pepsin-trypsin resistant gliadin (PT-gliadin).

5. The composition of any one of claims 1-4, wherein the composition is for alleviating symptoms of gluten-related disorder or treating gluten-related disorder in a subject.

6. The composition of any one of claims 1-5, wherein the subject is a human.

7. The composition of any one of claims 1-6, wherein the gluten-related disorder is celiac disease, non-celiac gluten sensitivity (NCGS), or gluten intolerance.

8. The composition of any one of claims 1-7, wherein the immunoglobulin is IgG.

9. The composition of any one of claims 1-8, wherein the immunoglobulin has a concentration of at least 1 mg/mL, at least 2 mg/mL, at least 3 mg/mL, at least 4 mg/mL, at least 5 mg/mL, at least 6 mg/mL, at least 7 mg/mL, at least 8 mg/mL, at least 9 mg/mL, at least 10 mg/mL, at least 15 mg/mL, at least 20 mg/mL, at least 25 mg/mL, at least 30 mg/mL, at least 35 mg/mL, at least 40 mg/mL, at least 45 mg/mL, or at least 50 mg/mL in the colostrum.

10. The composition of any one of claims 1-9, wherein the composition can inhibit or interfere absorption of the gluten or partially digested gluten in gastrointestinal (GI) tract of the subject.

11. The composition of any one of claims 1-10, wherein the immunoglobulin that specifically binds to the gluten or partially digested gluten is about 1-fold, about 2-fold, about 3-fold, about 4-fold, about 5-fold, about 6-fold, about 7-fold, about 8-fold, about 9-fold, about 10-fold, about 15-fold, about 20-fold, about 30-fold, about 40-fold, about 50-fold, about 60-fold, about 70-fold, about 80-fold, about 90-fold, or about 100-fold higher than that in a reference non-colostrum milk sample, or that in a reference colostrum sample that is produced by an animal without being immunized by the gluten or partially digested gluten.

12. The composition of any one of claims 1-11, wherein the composition is a lyophilized composition.

13. A food (e.g., medicinal food) or food additive prepared from the composition of any one of claims 1-12.

14. A method of immunizing an animal for production of colostrum, wherein the colostrum comprises an immunoglobulin that specifically binds to gluten or partially digested gluten, the method comprises:
a) administering the gluten or partially digested gluten to the animal, optionally with an adjuvant; and
b) collecting one or more milkings from the animal after parturition, wherein at least one of the one or more milkings comprises the colostrum.

15. The method of claim 14, wherein the animal is a non-human mammal.

16. The method of claim 14, wherein the animal is pregnant when administered with the gluten or partially digested gluten.

17. The method of any one of claims 14-16, wherein the animal is a cow (e.g., black Holstein, brown Holstein, or Jersey), buffalo, goat, reindeer, horse, sheep, camel, or yak.

18. The method of any one of claims 14-17, wherein the animal is a cow.

19. The method of any one of claims 14-18, wherein the animal is injected at least once, at least twice, at least three times, at least four times, at least five times, or at least six times prior to parturition.

20. The method of any one of claims 14-19, wherein the administering the gluten or partially digested gluten to the animal occurs at about 15 weeks, about 14 weeks, about 13 weeks, about 12 weeks, about 11 weeks, about 10 weeks, about 9 weeks, about 8 weeks, about 7 weeks, about 6 weeks, or about 5 weeks prior to parturition.

21. The method of any one of claims 14-20, wherein the collecting one or more milkings occurs within about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days about 4 days, about 5 days, about 6 days, or about 7 days after parturition.

22. A method of alleviating symptoms of a gluten-related disorder in a subject, comprising administering an effective amount of the composition of any one of claims 1-12, or the food or food additive of claim 13, to the subject.

23. A method for treating a gluten-related disorder in a subject, comprising administering an effective amount of the composition of any one of claims 1-12, or the food or food additive of claim 13, to the subject.

24. The method of claim 22 or 23, wherein the composition, the food, or the food additive is orally administered.

25. The method of any one of claims 21-24, wherein the composition, the food, or the food additive is orally administered with other food to the subject.

26. The method of any one of claims 21-25, wherein the composition, the food, or the food additive is orally administered to the subject before eating other food.

27. The method of any one of claim 25 or 26, wherein the other food comprises gluten.

28. The method of any one of claims 21-27, wherein the subject is a human.

29. The method of any one of claims 21-28, wherein the gluten-related disorder is celiac disease, non-celiac gluten sensitivity (NCGS), or gluten intolerance.

30. The method of any one of claims 21-29, further comprising providing gluten-free diet to the subject.
